(19) United States
(12) Patent Application Publication
(10) Pub. No.: US 2026/0004919 A1
(43) Pub. Date: Jan. 1, 2026

(54) AUTOMATIC DETECTION OF PATIENT AVAILABILITY IN A TELEHEALTH SYSTEM

(71) Applicant: TELADOC HEALTH, INC., Purchase, NY (US)

(72) Inventors: Luke Whitesides, South Lake Tahoe, CA (US); Shravan S. Rai, Goleta, CA (US); Paul C. McElroy, Goleta, CA (US); Sushil Pratap Bharati, Goleta, CA (US)

(21) Appl. No.: 18/758,998

(22) Filed: Jun. 28, 2024

(51) Int. Cl. G16H 40/20; G16H 10/60; G16H 40/67; G16H 80/00

(52) U.S. Cl. CPC G16H 40/20; G16H 10/60; G16H 40/67; G16H 80/00

(57) ABSTRACT

Telehealth consultations between a patient and a remote provider are useful in many settings, including inpatient care. Because patients in inpatient care are often moved in and out of their rooms during the day, inpatient care settings create unique challenges for remote providers and local care teams in terms of scheduling and completing telehealth consultations. Accordingly, disclosed herein is a system and method for automated detection and notification of a patient's call status in a telehealth network. The system automatically detects whether a patient is in their patient room. The system also monitors the status of a telehealth device in the patient room. The system displays the patient and device statuses to the remote provider so the remote provider can know when both the device and the patient are available for a consult. The system saves time by reducing calls made to empty patient rooms.

### TECHNICAL FIELD

The present disclosure pertains to telehealth systems and more specifically to automated detection of patient availability status.

### BACKGROUND

Telemedicine, telehealth, and/or virtual care is the provisioning of health care services using communications devices, such as personal computers (e.g., laptops, desktops, tablets, smartphones, etc.) and/or purpose-built devices (e.g., telemedicine carts, etc.) coupled to a communications network. Virtual care may involve a patient using a device to connect to and communicate with a remote health care provider, which may be a physician, clinician, counselor, coach, or trainer, to address health concerns of the patient. Virtual care may also be delivered in in-patient settings. In these cases, a remote provider may use a communication device to connect to another communication device located in a patient room, emergency room, operating room or other care location within a hospital or other healthcare facility. In-patient virtual care often involves a remote specialist consulting with local care providers, communicating with patients, and/or proctoring surgeries or other medical procedures.

Virtual care sessions may involve a two-way audiovisual conference between the remote provider and the patient and/or local provider, communication of medical data obtained from medical instruments coupled to a communication device, communication of health records between the local and remote sites, as well as communication of diagnoses, recommendations, and/or prescription information from the remote provider to the patient, local provider, and/or third parties such as electronic health records providers, insurance providers, and/or pharmacies.

Prior art telehealth devices in in-patient settings often take the form of wheeled carts equipped with video conferencing systems that may be moved from room to room. These carts may be moved by a bedside caregiver who also communicates and coordinates with a remote physician when the patient is available to participate in the telehealth consult. It is becoming increasingly common, however, for hospitals to equip patient rooms with dedicated in-room connected care devices. These are in-room, fixed telehealth devices that do not require a bedside caregiver to be in attendance with the patient or to move equipment around. These devices are often mounted to a wall and connected to a TV that is in the room so healthcare providers can virtually interact with a patient. Using these devices, a remote doctor, nurse, or other caregiver to connect to the telehealth device to conduct a 2-way audio/video session whenever the patient is available.

### SUMMARY

The present disclosure includes a method for notifying a remote healthcare provider of a call status in a telehealth system. The method comprises receiving, by a processor of a telehealth device, at least one image from a camera in a patient location. The at least one image is analyzed by the processor to determine whether a patient is present in the patient location. A patient presence value is updated by the processor based on the determination of whether a patient is present in the patient location. A call status value is then updated by the processor based on the patient presence value and a device status value associated with the telehealth device. The call status value is transmitted via a network for display at a remote device.

The present disclosure also includes a telehealth system that notifies a remote healthcare provider of a call status. The system comprises a processor in communication with a camera in a patient location. The processor is configured to analyze at least one image received from the camera to determine whether a patient is present in the patient location. The processor updates a patient presence value based on the determination of whether a patient is present in the patient location. The processor updates a call status value based on the patient presence value and a device status value associated with the telehealth device. The processor then transmits the call status value via a network for display at a remote device.

### BRIEF DESCRIPTION OF THE DRAWINGS

In order to describe the manner in which the above-recited and other advantages and features of the disclosure can be obtained, a more particular description of the principles briefly described above will be rendered by reference to specific embodiments thereof which are illustrated in the appended drawings. Understanding that these drawings depict only example embodiments of the disclosure and are not therefore to be considered to be limiting of its scope, the principles herein are described and explained with additional specificity and detail through the use of the accompanying drawings in which:

FIG. 1 is a schematic diagram of a telehealth system according to one embodiment of the present disclosure.

FIG. 2 is a schematic diagram of a patient room telehealth device according to one embodiment of the present disclosure.

FIG. 3 is a schematic diagram of an automated patient presence detection module according to one embodiment of the present disclosure.

FIG. 4A is a flowchart of a method for automated patient presence detection according to one embodiment of the disclosure.

FIG. 4B is a flowchart of a method for generating a call status value according to one embodiment of the present disclosure.

FIG. 5 illustrates a user interface for a remote provider according to one embodiment of the present disclosure.

FIG. 6 depicts an example computing system that may implement various systems and methods according to embodiments of the present disclosure.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without departing from the spirit and scope of the disclosure.

It should be understood at the outset that although illustrative implementations of one or more embodiments are illustrated below, the disclosed apparatus and methods may be implemented using any number of techniques. The disclosure should in no way be limited to the illustrative implementations, drawings, and techniques illustrated herein, but may be modified within the scope of the appended claims along with their full scope of equivalents.

A typical telehealth encounter may involve a patient and one or more remotely located physicians or healthcare providers. Devices located in the vicinity of the patient and the providers allow the patients and providers to communicate with each other using, for example, two-way audio and/or video conferencing.

A telepresence device may take the form of a desktop, laptop, tablet, smart phone, or any computing device equipped with hardware and software configured to capture, reproduce, transmit, and receive audio and/or video to or from another telepresence device across a communication network. Telepresence devices may also take the form of telepresence robots, carts, and/or other devices such as those marketed by Teladoc Health, Inc. of Purchase, New York, under the names VITA, LITE, VANTAGE, VICI, VIEWPOINT, XPRESS, and XPRESS CART. Telepresence devices may also take the form of stationary, in-room devices such as the Teladoc Health TV Pro, which may be installed in patient rooms and connected to existing audio/video hardware in the patient room, such as a TV or video display device. The physician telepresence device and the patient telepresence device may mediate an encounter, thus providing high-quality audio capture on both the provider-side and the patient-side of the interaction.

Telehealth consults with patients in in-patient care facilities can present unique challenges for remote providers. On any given day, a provider may have a list of remote consults to conduct with various patients located in different departments of a single facility or in various, geographically disparate facilities. Each patient to be seen remotely that day may be under the care of different care teams with different schedules and care routines. Patients in these facilities are often removed from their patient rooms for scans, therapy, or other procedures as needs arise and/or as equipment or staff becomes available. Also, some patients may be in their room but unavailable for other reasons, such as using the restroom. Thus, even if a particular patient's care team can schedule remote consultations between for the patient at a specific time of day, there is no guarantee the patient will actually be in their room and in the vicinity of the telehealth device the remote provider must connect to in order to see the patient. When a remote provider attempts to connect to an in-room device to consult with a patient and the patient is not present, the provider's time is wasted. When the remote provider contacts the absent patient's care team to locate the patient or reschedule the consult, the local care team's time is wasted. Missed appointments and rescheduling can lead to frustration, increased workload, and burn-out on the part of the provider and the local care team, ultimately reducing the quality of care the patient receives. In order alleviate this issue, the present disclosure provides a system and method for automatically determining the call availability status of each patient to be seen by a remote provider and providing an indication to the provider of which patients are in the vicinity of an in-room telehealth device and available for a consult with the remote provider. This way, when a patient requiring a consult is not currently available, the remote provider can simply choose another patient on their schedule that is currently available for a call, thereby reducing the wasted time and other issues associated with missed calls and rescheduling.

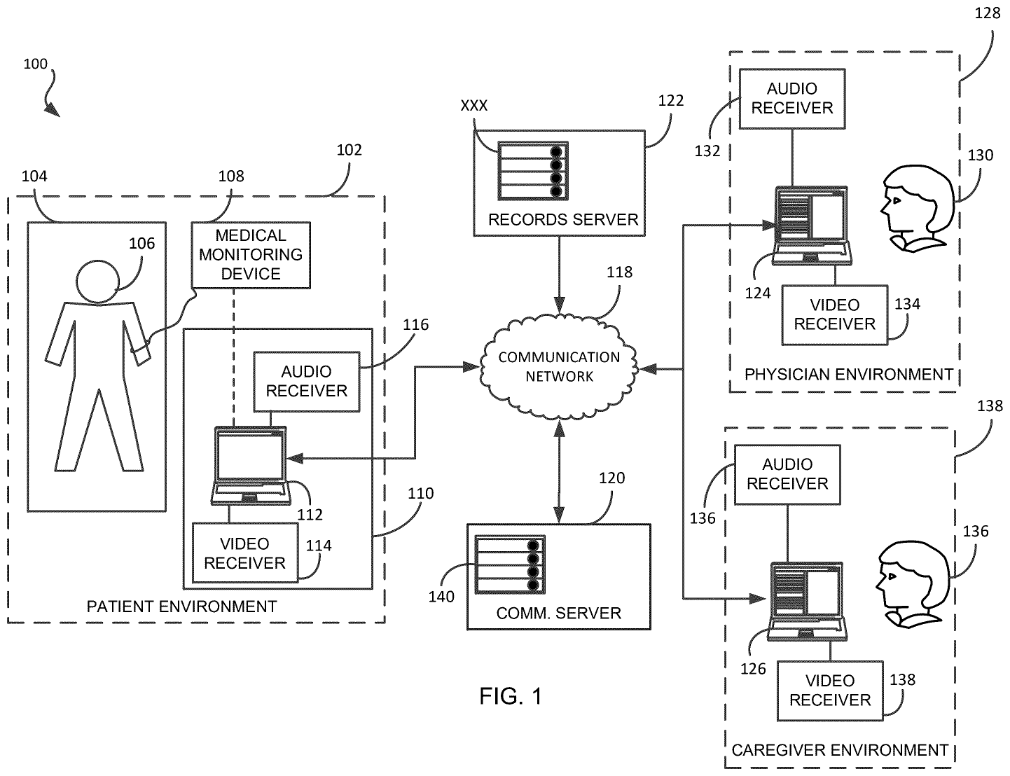

FIG. 1 illustrates a telehealth system 100 automatically determining the call availability status of a patient located in a patient environment 102, such as in-patient care facility. The patient environment 102 may include a patient bed 104, or other furniture, such as a chair, where the patient is situated when present in the room. The patient bed 104 may or may not have a patient 106 in it at any given time. The patient 106 may be connected to one or more medical monitoring devices 108 to monitor the patient's vital signs or other health indicators.

The patient environment 102 may also include an in-room telehealth device 110, which may include at least a computing device 112, a video receiver 114 in the form of one or more cameras, and an audio receiver 116 in the form of one or more microphones. The computing device may take the form of any computing device capable of performing the automatic patient presence detection function described in detail below. Examples of computing devices include laptops, desktops, smart phones, as well as dedicated telehealth devices such as the TV PRO marketed by Teladoc Health, Inc.

The system 100 may also include a communications network 118 that connects the telehealth device 110 to a communication server 120, a records server 122, a provider device 124, and a caregiver device 126.

The provider device 124 may be operated by a physician 130 located in a physician environment 128, such as a hospital, the physician's office, home, car, or any other location with network connectivity. The physician device may be any telepresence-capable device as discussed above and, like the telehealth device 110 in patient environment 102, include or be coupled to an audio receiver 132 and a video receiver 134.

The caregiver device 126 may be operated by a caregiver 136 in a caregiver environment 138. The caregiver 136 may be a nurse or other caregiver to the patient 106. The caregiver environment 138 may be a nurses' station within the facility that houses the patient environment 102 or any location including one or more caregivers tasked with monitoring the patient 106 and possibly other patients. Like the provider device 124, the caregiver device 126 may be any telepresence device as discussed above and, like the telehealth device 110 in patient environment 102, include or be coupled to an audio receiver 136 and a video receiver 138.

The communication server 120 may be one or more remotely connected computer servers 140 that provide various computing functions to support the functions of the telehealth system. The communication server may, for example, facilitate call setup, manage user authentication and permissions, monitor telehealth devices 110 and their statuses, report device status and usage information, deploy software and firm updates, as well as provide communications services such as firewall traversal and/or ICE/STUN/TURN protocols. In some examples, the communication server 120 may include a virtual server and the like provided over a cloud-based service, as will be understood by a person having ordinary skill in the art.

Both the physician 130 and the caregiver 136 may retrieve and review an electronic medical record (“EMR”) and other medical data related to the patient 106 from a networked records server 122. The records server may receive medical data directly from the medical monitoring device 108. The records server 122 can be a computer server 142 remotely connected to the telehealth device 110, medical monitoring device 108, provider device 124 and/or the caregiver device 126 via the communication network 118 or may be onsite with the physician 106, caregiver 136, or the patient 106. Either the records server 122 or communication server 120 may also provide a scheduling service that allows the patient 106, provider 130, and/or caregiver 136 to schedule telehealth visits between patient 106 and provider 130. The schedule may be visible to one or more of the parties via a browser or dedicated app running on the local device. When the appointment nears, one or more of the parties may receive a reminder notification on their device. The reminder may include a link that the local user can activate to initiate the telehealth call.

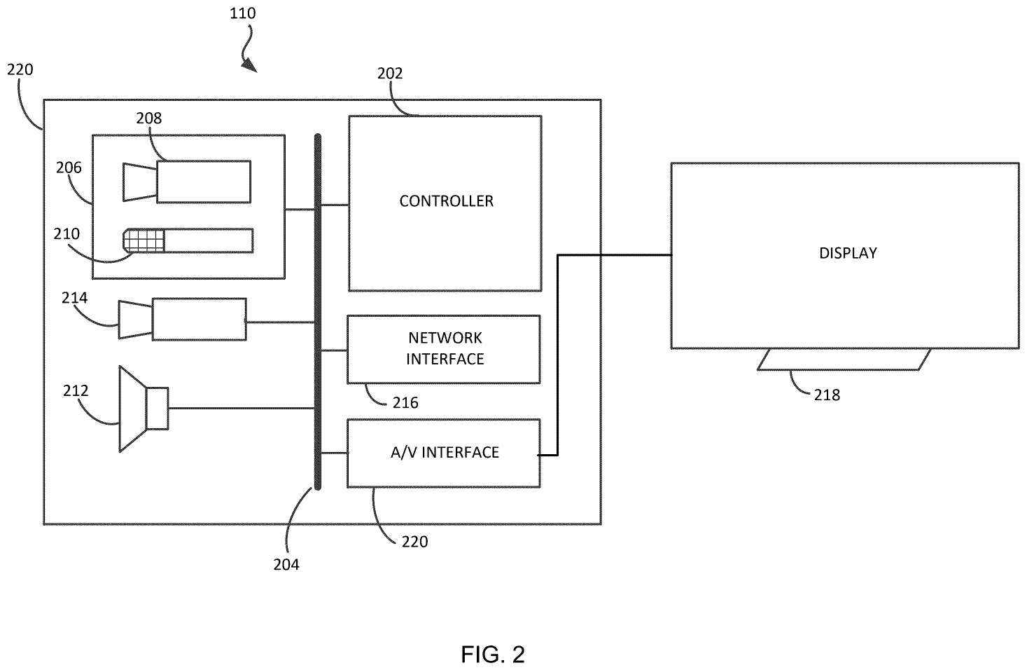

FIG. 2 is a schematic diagram of a telehealth device 110 in accordance with one embodiment of the present disclosure. The device 110 may include a housing 220 that contains a controller 202 coupled to a bus 204. The bus 204 may be coupled to a pan-tilt-zoom (“PTZ”) camera system 206 capable of moving a video camera 208 and a microphone 210 together around a pan axis and tilt axis. The PTZ system 206 may be remotely controlled by the remote provider during a telehealth consult to adjust the field of view of the camera 208 to provide an optimal view of the patient. The camera 208 may have a high optical zoom factor, e.g., 30X, that can be remotely controlled to allow the provider to examine the patient's skin, eyes, etc., at a resolution that meets or exceeds that of an unassisted eye during an in-person visit. Microphone 210 is mounted on PTZ system 206 to face the same direction as the camera 208 and moves (e.g., pans and tilts) with the camera. Microphone 210 may be a directional microphone designed to isolate on-axis sound emanating from the direction the microphone is facing (i.e., the field of view of the camera) and suppresses off-axis sound emanating from elsewhere. This arrangement can produce a higher quality audio experience for the provider when listening to the patient during the telehealth consult.

The telehealth device 110 may also include a speaker 212 and a stationary camera 214 coupled to the bus 204. The speaker may reproduce sound captured by a microphone of the provider or caregiver devices during the telehealth consult, allowing the patient to hear the provider or caregiver. The stationary camera 214 may provide a wide field of view that allows the remote caregiver to visually monitor the entire patient room. The stationary camera 214 may also include an infrared filter that allows the caregiver to monitor the patient room when the room is dark.

The telehealth device 110 may include a network interface 216 that transmits audio, video, status, and other data from the other components of the telehealth device 110 to other elements of the telehealth system via the network. Network interface 216 may also receive audio, video, control, status, and other data from other components of the telehealth system via the network. Network interface 216 may include a wired and/or wireless connection to a local area network (LAN) that serves the patient environment. This LAN may provide access to the Internet via an Internet service provider. By way of example, network interface 216 may transmit video from camera 208 and audio from microphone 210 to the remote provider's device via the Internet. Likewise, the network interface 216 may receive audio, video, and control data from the provider device via the Internet. The network interface may also transmit status data from the controller to the communication server via the network.

The telehealth device 110 may be coupled to a TV or display device 218 via an A/V interface 220 coupled to the bus 204. A/V interface 220 may relay video data received from the provider device, via the network interface, to the display device 218 for display. By way of example, the display device 218 may display video of the provider's face for the patient to view during the telehealth consult.

The telehealth device 110 may include a power supply (not shown) coupled to a battery, power cord, or both. The power supply may be coupled to bus 204 and provide electrical power to the other components of the device 110 via the bus 204 or dedicated power connections.

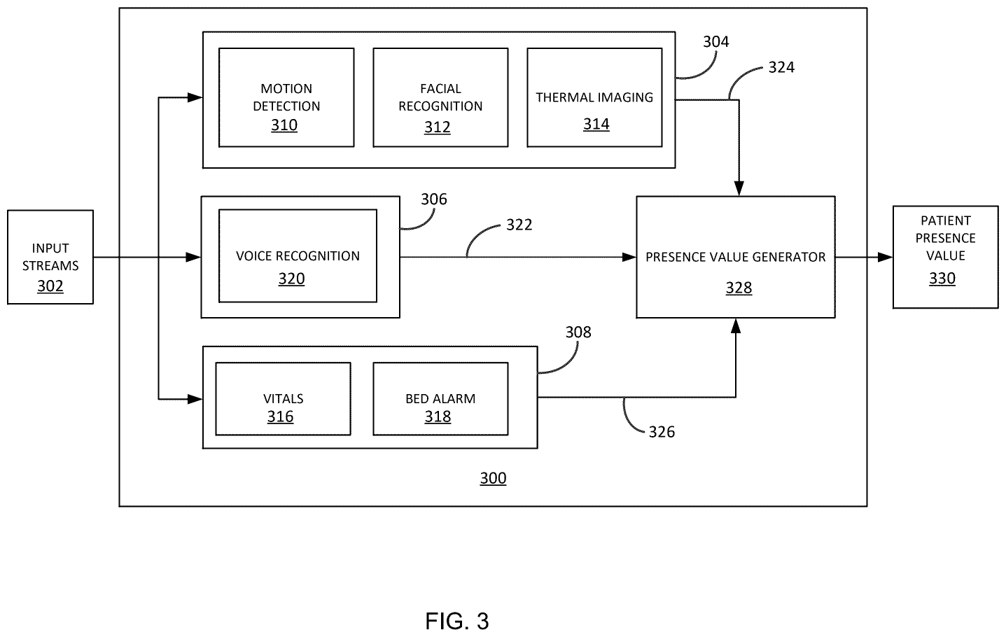

FIG. 3 is a schematic diagram of an automated patient presence detection module 300 that may be executed on the controller 202 of telehealth device 110 (see FIG. 2). The module 300 may include several modules for analyzing different data types that may be available in the input streams 302, including an image analysis module 304, an audio analysis module 306, and a monitoring device module 308.

Input streams 302 may include video data, audio data, and/or or any other available data. Other data may include data available from a vital signs monitors, bed alarms or other medical monitoring device data. The input streams 302 may undergo one or more modes of analysis depending on the availability of each data type, user preferences, organizational policies, laws, regulations, and the like. For example, if only audio is present in the stream 302, the module 300 may only use the audio analysis module 306. If, however, the stream 302 contains both audio and video data, the module may employ both the image analysis module 304 and the audio analysis module 306, etc.

Within each module may be one or more algorithms that perform a mode of patient presence detection specific to that module. For example, image analysis module 304 may include one or more of a motion detection algorithm 310, a facial recognition algorithm 312, and a thermal imaging algorithm 314, if thermal imaging is available. Similarly, the medical monitoring device module 308 may include algorithms that can detect patient presence based on vitals monitoring data 316 and or data from a patient bed alarm 318, which may use a weight sensor to detect whether the patient is currently occupying the bed. The audio analysis module 306 may include a voice recognition algorithm 320 that can determine whether the patient is present by analyzing the audio data received from a microphone in the room to detect the patient's vocal signature. Each algorithm may output one or more confidence scores 322, 324, 326 representing the likelihood that the patient is present in the patient room or otherwise available for a call based on the algorithm's analysis of its respective input data. A presence value generator 328 receives each of the one or more confidence scores 322, 324, 326 and combines them according to a statistical model that applies predetermined weights to each confidence score to produce a binary patient presence value 330, which may have a value of TRUE or FALSE.

Each or any of the algorithms in the image analysis module 304 may include an object detection layer that can identify objects or areas of interest within the video stream. Areas of interest may include the patient bed, chairs, tables, doorways, and medical equipment present in the patient room. An area of interest may also include a human being that is detected in the stream.

The motion detection algorithm 310 may be configured to measure the magnitude and recency of motion in the received video stream. By way of example, when the motion detection algorithm 310 detects motion in the video stream, it may compare the area in which the motion occurred to a size threshold to determine whether the movement was that of a human being or something smaller. If the area of motion is larger than the threshold, the algorithm 310 may output a high confidence score indicating that the patient is likely present in the room. When the detected motion ceases, the confidence score output by the algorithm may decay at a predetermined rate to indicate a decreasing likelihood that the patient is present in the video.

The motion detection algorithm 310 may be configured to identify an object (such as a human being) and track the motion of the object relative to another object or area of interest. The motion detection algorithm 310 may treat the measured motion differently depending on the type of object identified and/or the proximity and direction of motion relative to another object or area of interest. For example, if the motion detection algorithm 310 detects and tracks a human-type object moving from the patient bed to the patient room door and subsequently detects no further motion, the algorithm 310 may output a lower confidence score and/or decay the last confidence score more rapidly based on the assumption that the patient has likely left the room. On the other hand, if the algorithm detects a human moving from the door to the patient bed and subsequently detects no additional motion, the algorithm 310 may output a high confidence and/or decay the last confidence score more slowly based on the assumption that the patient has gotten into bed. This approach is especially useful in circumstances where a patient in the room is considered available for a remote consult even when they are resting in bed.

The object detection may be performed using one or more machine learning algorithms that have been trained to identify standard objects. In addition, the object detection may also apply logic or rules specific to a hospital room and/or particular use cases. For example, the algorithm may be configured to detect one or more persons in the room and identify one of the persons as a patient by determining which person is in the hospital bed. The algorithm may also then continuously track that person's identity as the patient over time using a tracking algorithm. In one embodiment, the object detection algorithm may be implemented as a convolutional neural network such as YOLO (You Only Look Once). The tracking algorithm may be implemented as Vision Transform (ViT) model. The object detection and tracking functions may also be implemented using other suitable algorithms as will be known to those of skill in the art.

The image analysis module 304 may also employ a facial recognition algorithm 312 that can recognize faces within an image and even identify the person associated with a detected face where a database with information linking a specific set of facial features to the identity of the person is available. Facial recognition module 312 may be useful for determining whether the patient is present even when they have not moved enough to confirm their presence using the motion detection module 310 alone. The facial recognition module 312 may be trained to identify the patient's face as well as the faces of hospital staff when the necessary training data is available (such as a database of photographs of patients and/or staff). In addition, the facial recognition algorithm may learn to recognize the face of the patient as opposed to staff by noting the position and duration of the face relative to certain areas of interest. For example, the facial recognition algorithm may be configured to recognize a face detected at the head of the patient bed for long periods of time as the face of the current patient associated with the patient room.

In addition, where the facial recognition module can identify faces it detects in the video, it can be used to distinguish between the patient and other people who may be present in the room, such as a doctor, nurse, or other staff. By way of example, even if the motion detection 310 module detects a person in the room and outputs a high confidence score, the facial recognition may output a low confidence score if the detected face is not the patient's face or is the face of a hospital staff member. Thus, the facial recognition algorithm can be useful for improving the accuracy of the resultant patient presence value.

Certain modes of analysis may not be performed even when the necessary data is available. For example, if patient preferences or organization policies prohibit the use of facial recognition technology, the facial recognition 312 would not be performed on any incoming video data.

The module 300 may reside on a controller or computing device located elsewhere (e.g., communication server 120, FIG. 1, or any other suitable computing device in communication with the telehealth device 110). Images of the patient may, however, constitute personally identifiable information or protected health information, and thus certain laws and or organizational policies may prevent such images from being transmitted to another location without the patient's explicit consent, which may not always be possible to obtain. Thus, in a one embodiment of the invention, the automated patient presence detection is performed on the device located in the patient's room, thereby eliminating the need to transmit images of the patient over a network. In this embodiment, only an indication of whether a patient is present and or available for a call need be transmitted over the network.

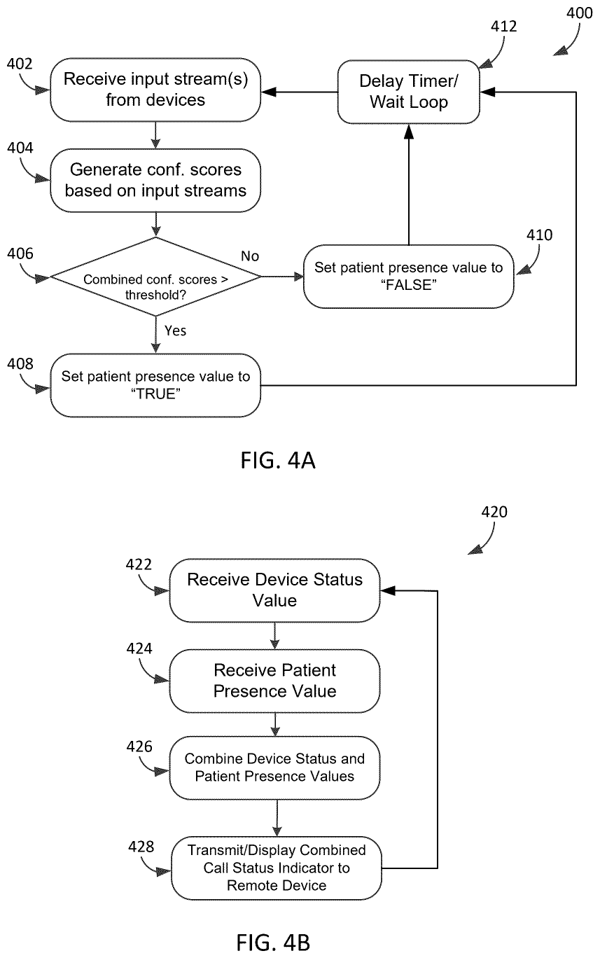

FIG. 4A is a flow chart illustrating a process 400 performed by the automated patient presence detector module 300 executing on the controller 202 of the telehealth device 110 described above. The process 402 begins at step 402 when one or more input data streams are received from the devices in the patient room. As discussed above, these input streams may include video from one or more cameras, audio from one or more microphones, and monitoring data from one or more patient monitoring devices.

At step 404, the input streams are analyzed by the appropriate modules and/or algorithms as discussed above with respect to FIG. 3. At step 404, depending on the availability of the input data, one or more of the modules and/or algorithms may generate one or more confidence score based on its respective analysis of the corresponding data stream.

At step 406, the one or more confidence scores generated at step 404 are combined according to a weighted statistical model and compared to a predetermined threshold. If the combined score meets or exceeds the predetermined threshold, the process proceeds to step 408 and the controller sets the patient presence value to TRUE. If the combined confidence score falls below the predetermined threshold, the process proceeds to step 410 where the controller sets the patient presence value to FALSE.

After either of step 408 or 410, the process proceeds to step 412 at which point the controller executes a delay timer or wait loop for a predetermined time interval before returning to step 402 to repeat the process. Thus, the controller may be configured to repeat process 400 at a specified rate as determined by the wait loop. For example, the controller may be configured to repeat the process 400 continuously by setting the wait loop equal to zero. Alternatively, the wait loop may be set to cause the process to repeat every 30 seconds, 60 seconds, two minutes, five minutes, 15 minutes, 30 minutes, 1 hour, etc. The duration of the wait loop can be set to any time interval suitable for determining the patient's presence within the context of assessing the patient's readiness to participate in a telehealth consultation.

FIG. 4B illustrates a process 420 for generating a call status indicator to be displayed by a user interface of the remote provider device. The call status indicator is a combination of a device status indicator and the patient presence value calculated in process 400, described above with respect to FIG. 4A. The device status indicator may indicate whether the telehealth device in the patient's room is offline, ready, or busy. The device status indicator may be set to OFFLINE if, for any reason, the communications server is unable to establish or maintain a connection with the telehealth device in the patient room. The device status indicator may be set to READY if the device is online and capable of receiving a call. The device status indicator may be set to BUSY if the telehealth device is online but currently in a call or otherwise unable to receive a new call.

The call status indicator process 420 may be performed at the telehealth device 110, the communication server 120, or a remote device 124 and/or 126. Process 420 begins at step 422 where a controller on the device executing the process 420 receives or otherwise reads the device status value of the telehealth device associated with the patient room. At step 424, the controller receives or otherwise reads the patient presence value calculated in process 400 described above with respect to FIG. 4A.

At step 426, the received device status value and the patient presence value are combined to generate a call status value. The device status value and patient presence value can be combined to form the call status value in any number of ways known to those of skill in the art. By way of example, the device status and patient presence values may be combined using a lookup table as illustrated below in TABLE I.

TABLE I

Patient Presence Value

| | | |
|---|---|---|
| Call Status Value Mapping | FALSE | TRUE |

| | | | |
|---|---|---|---|
| Device Status | OFFLINE | 0 | 0 |
| Indicator | BUSY | 1 | 1 |
| | READY | 2 | 3 |

At step 428, the call status indicator calculated in step 426 is either transmitted to the remote device for display or, if process 420 is performed at the remote device, displayed at the remote device.

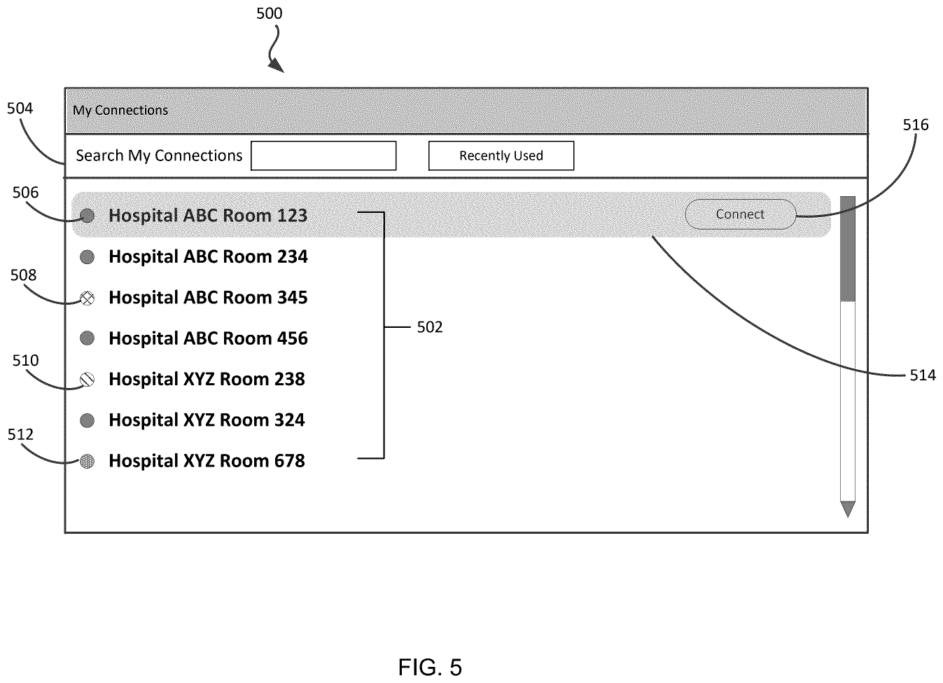

As discussed further below with respect to FIG. 5, the call status indicator value may be mapped to a display scheme that allows the user of the remote device to quickly apprehend whether both the patient device and the patient themselves are ready to receive a call.

FIG. 5 illustrates an example of a user interface 500 that may be displayed on a remote device, such as the provider device or the caregiver device shown in FIG. 1. The interface 500 may include a list of names care locations 502. Care locations may be various rooms at various hospitals. Each care location in the list 502 represents a location equipped with a device such as telehealth device 110 that described in FIG. 1 and that the user of the remote device is authorized to connect to.

The interface 500 may include a search and/or filter function bar 504 that allows the user to enter text to search for one or more care location names or sort or filter the list of names according to the recency of connection or use. The interface 500 may also include a cursor 514 that indicates which care location is currently selected and a CONNECT button 516 that, when selected, causes the remote device to attempt to establish a connection with the telehealth device at the corresponding care location.

Each care location name in the list 502 may have an associated call status indicator next to it, such as those represented by elements 506, 508, 510, and 512. Each call status indicator may have a distinct visual characteristic corresponding to the call status value for that care location. By way of example, and referring also to TABLE I above, the color or fill pattern of call status indicator 506 may represent call status value “3” in TABLE I, meaning the device is online, available for a call, and the patient is present. In the same example, the color or fill pattern of call status indicator 508 may represent call status “0”, meaning the device is offline. Similarly, the color or fill pattern of indicator 510 may represent call status “1”, meaning that the device is online but is currently busy in another call. And the color or fill pattern of indicator 512 may represent call status value “2”, meaning that the device is online and available to receive a call, but the patient is not present.

It is to be appreciated that the call status indicators in FIG. 5 are illustratively only. One of ordinary skill in the art will appreciate that the call status value of each care location can be represented in the interface 500 in any number of ways. For example, instead of distinct indicators associated with each care location, the text that forms the name of the care location or the background fill/color associated with each care location could be altered to indicate the call status value for that location. In another embodiment, the search and filter function bar could include a filter function to only display care locations with specific call status values. That way, the remote provider could configure the interface 500 to display only those call locations that are available with the patient present, and/or only those locations that are available with patient present or patient not present.

In yet another embodiment, the remote device may receive the device status indicator and the patient presence indicator and display them as two separate indicators associated with each care location.

The representation of the different call status values may be useful to a remote provider attempting to reach a patient. For example, if the remote provider intends to call a care location indicated as either busy or available but patient not present, the provider may simply wait until the call status indicator for that location changes. If, however, the call status indicator for that care location indicates that the device is offline or unreachable, the provider may contact a help desk or the care team at the corresponding care facility to ask that the device be brought online. In general, a system and method according to the present disclosure, which allows a remote provider to quickly and easily apprehend which patients, among the many patients the provider may need to see, can actually be seen at any given moment, saves time at least on the part of the provider, if not local care teams as well, and can ultimately result in a better telehealth experience for all involved and higher quality of care for patients. Importantly,

An advantage of a system and method in accordance with the present disclosure is that the call status, or device status and patient status indicators, are proactively transmitted to each provider device and periodically refreshed. This eliminates the need of the provider to select a care location from the list and attempt to connect to the device in that care location before receiving information about the status of the device, the patient, or both. That is, the provider can instantly see which care locations (or patients) in the provider's list are ready for a call, rather than the provider attempting to connect and then receiving a “call declined” message or the like.

Moreover, in one embodiment, the provider interface is configured to allow the provider to attempt to connect to a care location even if the call status and/or patient status and device status indicators do not indicate the care location is ready for a call. For example, the CONNECT button 516 may be displayed next to any selected care location and cause the provider device to attempt to connect to the in-room device at the corresponding care location regardless of whether the received status information indicates that the patient or the device is available. This may be useful in situations where the provider is needed at a particular care location but a technical issue causes incorrect call status information to be displayed at the provider interface.

After the provider selects to connect to a particular care location, the in-room at the care location may be configured to display or playback a prompt that invites a person at the care location to accept or decline the call from the provider. However, in an in-patient setting, this call flow may be undesirable. For example, there may be situations when the patient is unable to interact with the in-room device to accept the call. Therefore, in another embodiment, the in-room telehealth device is configured to accept the incoming call from the provider automatically and begin transmitting video and/or or audio to the provider, as well as displaying and/or reproducing video and audio received from the provider device. Though this configuration may be undesirable for consumer video conferencing products designed for use in homes and offices, it may be preferable in certain healthcare contexts for the reasons discussed above. In addition, the system may be configured automatically accept the call in an audio-only mode. This way, the video from the care location may not be transmitted until the provider has obtained audible consent from the patient or care team to do so. In general, however, in the context of a healthcare system in which providers have obtained consent to treat patients, and patients may be limited in their abilities, it is advantageous for the provider to be able to initiate a virtual visit with each patient without requiring the patient to interact with the in-room device. In addition, although the above described system is discussed primarily for use in in-patient healthcare settings, it may also be useful in certain homecare settings where a patient is at located at home but requires regular virtual care visits. In this context, the automatic call accept function may be configured to best suit the needs and limitations of any specific patient.

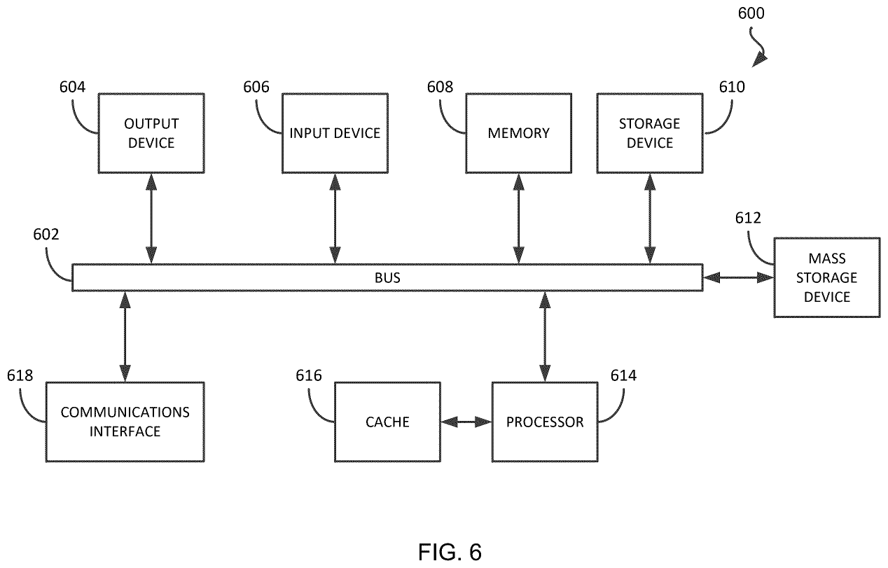

FIG. 6 depicts an example computing device or computer system 600 that may implement various systems and methods discussed herein. The computer system 600 includes one or more computing components in communication via a bus 602. In one implementation, the computer system 600 includes one or more processors 614. Each processor 614 may include one or more internal levels of cache 616, as well as bus controller or bus interface unit to direct interaction with a bus 602.

A memory 608 may include one or more memory cards and control circuits (not depicted), or other forms of removable memory, and may store various software applications including computer executable instructions, that when run on the processor 614, implement the methods and systems set out herein. Other forms of memory, such as a mass storage device 610, may also be included and accessible, by the processor (or processors) 614 via the bus 602.

The computer system 600 may further include a communications interface 618 by way of which the computer system 600 can connect to networks and receive data useful in executing the methods and system set out herein as well as transmitting information to other devices. The computer system 600 may include an output device 604, such as graphics card or other display interface by which information can be displayed on a computer monitor. The computer system 600 can also include an input device 606 by which information is input. Input device 606 can be a mouse, keyboard, scanner, and/or other input devices as will be apparent to a person of ordinary skill in the art.

The system set forth in FIG. 6 is but one possible example of a computer system that may employ or be configured in accordance with aspects of the present disclosure. It will be appreciated that other non-transitory tangible computer-readable storage media storing computer-executable instructions for implementing the presently disclosed technology on a computing system may be utilized.

The described disclosure may be provided as a computer program product, or software, that may include a computer-readable storage medium having stored thereon instructions, which may be used to program a computer system (or other electronic devices) to perform a process according to the present disclosure. A computer-readable storage medium includes any mechanism for storing information in a form (e.g., software, processing application) readable by a computer. The computer-readable storage medium may include, but is not limited to, optical storage medium (e.g., CD-ROM), magneto-optical storage medium, read only memory (ROM), random access memory (RAM), erasable programmable memory (e.g., EPROM and EEPROM), flash memory, or other types of medium suitable for storing electronic instructions.

The description above includes example systems, methods, techniques, instruction sequences, and/or computer program products that embody techniques of the present disclosure. However, it is understood that the described disclosure may be practiced without these specific details.

While the present disclosure has been described with references to various implementations, it will be understood that these implementations are illustrative and that the scope of the disclosure is not limited to them. Many variations, modifications, additions, and improvements are possible. More generally, implementations in accordance with the present disclosure have been described in the context of particular implementations. Functionality may be separated or combined in blocks differently in various embodiments of the disclosure or described with different terminology. These and other variations, modifications, additions, and improvements may fall within the scope of the disclosure as defined in the claims that follow.

We claim:

1. A method for notifying a remote healthcare provider of a call status in a telehealth system, the method comprising:
receiving, by a processor of a telehealth device, at least one image from a camera in a patient location;
analyzing the at least one image to determine whether a patient is present in the patient location;
updating a patient presence value based on the determination of whether a patient is present in the patient location;
updating a call status value based on the patient presence value and a device status value associated with the telehealth device; and
transmitting the call status value via a network for display at a remote device.

2. The method of claim 1, wherein analyzing the at least one image includes performing [computer vision method(s)/technique(s)] on the at least one image.

3. The method of claim 1, wherein the device status value indicates whether the telehealth device is available for a call.

4. The method of claim 1, wherein updating the call status value includes performing an operation on the patient presence value and the device status value.

5. The method of claim 1, wherein the remote device displays the call status in association with at least one of a patient identifier and a patient location identifier.

6. The method of claim 5, wherein the call status is displayed in a list comprising a plurality of patient identifiers or patient location identifiers.

7. The method of claim 6, wherein a user of the remote device selects one of the plurality of patient identifiers or patient location identifiers to initiate a call with the telehealth device at the patient location.

8. The method of claim 7, wherein, during the call, video received from the remote device is displayed on a display device at the patient location.

9. The method of claim 1, wherein determining whether a patient is present in the at least one image includes determining whether a person is present in the at least one image and, when a person is determined to be present in the at least one image, determining whether the person is a patient.

10. The method of claim 9, wherein determining whether the person is a patient includes analyzing at least one of facial features, clothing, and the position of the person within the patient location.

11. A telehealth device that notifies a remote healthcare provider of a call status, the device comprising:
a processor in communication with a camera in a patient location, wherein the processor is configured to:
analyze at least one image received from the camera to determine whether a patient is present in the patient location;
update a patient presence value based on the determination of whether a patient is present in the patient location;
update a call status value based on the patient presence value and a device status value associated with the telehealth device; and
transmitting the call status value via a network for display at a remote device.

12. The device of claim 11, wherein the processor is configured to perform [computer vision technique] on the at least one image.

13. The device of claim 11, wherein the device status value indicates whether the telehealth device is available for a call.

14. The device of claim 11, wherein the processor is configured to update the call status value by performing an operation on the patient presence value and the device status value.

15. The device of claim 11, wherein the remote device is configured to display the call status in association with at least one of a patient identifier and a patient location identifier.

16. The device of claim 15, wherein the remote device is configured to display the call status in a list comprising a plurality of patient identifiers or patient location identifiers.

17. The device of claim 16, wherein a user of the remote device selects one of the plurality of patient identifiers or patient location identifiers to initiate a call with the telehealth device at the patient location.

18. The device of claim 17, wherein, during the call, video received from the remote device is displayed on a display device at the patient location.

19. The device of claim 11, wherein determining whether a patient is present in the at least one image includes determining whether a person is present in the at least one image and, when a person is determined to be present in the at least one image, determining whether the person is a patient.

20. The device of claim 19, wherein determining whether the person is a patient includes analyzing at least one of facial features, clothing, and the position of the person within the patient location.
